(19) United States
(12) Patent Application Publication
(10) Pub. No.: US 2023/0255865 A1
(43) Pub. Date: Aug. 17, 2023

(54) Oral Care Compositions

(71) Applicant: Colgate-Palmolive Company, New York, NY (US)

(72) Inventors: Guisheng PAN, Philadelphia, PA (US); Lin FEI, Kendll Park, NJ (US); Suman CHOPRA, Monroe, NJ (US); Pooja KULKARNI, Plairnsboro, NJ (US)

(73) Assignee: Colgate-Palmolive Company, New York, NY (US)

(21) Appl. No.: 18/043,753

(22) PCT Filed: Aug. 30, 2021
(86) PCT No.: PCT/US2021/048239

§ 371 Date: Mar. 2, 2023

Related U.S. Application Data
(60) Provisional application No. 63/073,784, filed on Sep. 2, 2020.

(51) Int. Cl. A61K 8/36; A61Q 11/00

(52) U.S. Cl. CPC A61K 8/361; A61Q 11/00; A61K 2800/48; A61K 2800/28; A61K 2800/92

(57) ABSTRACT

Described herein are high water oral care composition comprising an orally acceptable vehicle; a thickening system; one or more abrasives; and one or more gelling agents, wherein the one or more gelling agents comprise a salt of a fatty acid; and wherein the salt is a monovalent salt, a divalent salt, or a combination thereof. Also disclosed are methods of making and using the same.

### CROSS-REFERENCE TO RELATED APPLICATION

This application claims the benefit of priority from U.S. Provisional Application No. 63/073,784, filed Sep. 2, 2020, the contents of which are hereby incorporated herein by reference in their entirety.

### BACKGROUND

Abrasives comprising calcium phosphate salts, such as calcium pyrophosphate, dicalcium phosphate dihydrate (sometimes referred to as DiCal), tricalcium phosphate, and calcium polymetaphosphate are useful in dentifrice materials. In addition to providing an abrasive action, which cleans the teeth, such salts also provide a source of calcium and phosphate which can help build and repair the teeth.

Calcium salts such as calcium carbonate, precipitated calcium carbonate (PCC), and natural calcium carbonate (NCC), are also commonly used as abrasives. However, it is difficult to stabilize high water toothpaste containing less than 30% PCC or NCC abrasive.

It would therefore be desirable to provide an oral care composition which comprises a lower levels of calcium salt abrasive, but which also maintains the stability and beneficial physical attributes of higher calcium salt containing compositions.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

Applicants have discovered that utilization of certain fatty acid salts is effective as a gelling agent for oral care products, particularly for oral care products containing a high water content. Thus, in one embodiment, the invention is a high water oral care composition comprising an orally acceptable vehicle; a thickening system; one or more abrasives; and one or more gelling agents, wherein the one or more gelling agents comprise a salt of a fatty acid and wherein the salt is a monovalent, divalent, or a combination thereof.

In at least one embodiment, the fatty acid is a C₁₂-C₃₂ fatty acid. In a further embodiment, the fatty acid is a saturated fatty acid. In other embodiments, the fatty acid is an unsaturated fatty acid. In certain embodiments, the unsaturated fatty acid has an iodine value of less than 5 or less than 1. In certain embodiments, the unsaturated fatty acid is a C₁₆₋₁₈ fatty acid and has an iodine value of less than 5 or less than 1. In certain embodiments, the fatty acid is selected from lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, linoleic acid, arachidonic acid, palmitoleic acid, oleic acid, and a combination of two or more thereof. In certain embodiments, the fatty acid is selected from palmitic acid, stearic acid, and a combination thereof.

In certain embodiments, the monovalent salt of a fatty acid is selected from sodium salt, potassium salt, lithium salt, and a combination of two or more thereof. In certain embodiments, the monovalent salt of a fatty acid is selected from sodium palmitate, sodium stearate, potassium palmitate, potassium stearate, and a combination of two or more thereof. In certain embodiments, the one or more gelling agents are present in an amount of from about 0.5 weight % to about 4.5 weight %, based on the total weight of the oral care composition.

In certain embodiments, the orally acceptable vehicle is selected from glycerin, sorbitol, xylitol, propylene glycol, polyethylene glycol, and combinations of two or more thereof. In certain embodiments, the orally acceptable vehicle is present in an amount of from about 50 weight % to about 70 weight %, based on the total weight of the oral care composition.

In certain embodiments, the thickening system comprises colloidal silica, fumed silica, carrageenan, sodium carboxymethylcellulose, or a combination thereof. The thickening system may be present in an amount of from about 2 weight % to about 5 weight %, 2.5 weight % to about 4.5 weight %, or 3 weight % to about 4 weight %, based on the total weight of the oral care composition.

In certain embodiments, the one or more abrasives comprise calcium salt, silica, or sodium metaphosphate. In certain embodiments, the calcium salt is selected from tricalcium phosphate, hydroxyapatite, dicalcium phosphate dihydrate, anhydrous dicalcium phosphate, calcium pyrophosphate, natural calcium carbonate, precipitated calcium carbonate, and mixtures thereof. In certain embodiments, the silica is selected from precipitated silica, hydrated silica, and mixtures thereof. The one or more abrasives may be present in an amount of from about 15 weight % to about 25 weight %, based on the total weight of the oral care composition.

In certain embodiments, the oral care composition further comprises a fluoride ion source. The fluoride ion source may be selected from stannous fluoride, sodium fluoride, potassium fluoride, sodium monofluorophosphate, fluorosilicate salts, such as sodium fluorosilicate and ammonium fluorosilicate, amine fluoride, ammonium fluoride, and combinations thereof. The fluoride ion source may be present in an amount of from about 0.01 weight % to about 5.0 weight %, 0.01 weight % to about 3.0 weight %, or 0.01 weight % to about 1.0 weight %, based on the total weight of the oral care composition.

In certain embodiments, the oral care composition is substantially free of fatty alcohols. In certain embodiments, the oral care composition comprises fatty alcohols in an amount of less than 5.0 weight %, less than 3.0 weight %, less than 1.0 weight %, less than 0.1 weight %, less than 0.05 weight %, less than 0.01 weight %, less than 0.005 weight %, or less than 0.0001 weight %, based on the total weight of the oral care composition.

In certain embodiments, the oral care composition comprises water in an amount of at least 25 weight %, at least 30 weight %, at least than 35 weight %, at least than 40 weight %, or at least than 45 weight %, based on the total weight of the oral care composition. In certain embodiments, the oral care composition comprises water in an amount of from about 20 weight % to about 50 weight %, 25 weight % to about 45 weight %, 30 weight % to about 45 weight %, or 35 weight % to about 45 weight %, based on the total weight of the oral care composition.

In certain embodiments, the oral care composition further comprises one or more surfactants. The one or more surfactants may be selected from anionic surfactants. The one or more anionic surfactants may be selected from sodium lauryl benzene sulfonate, dodecyl sodium sulfoacetate, N-2-ethyl laurate potassium sulfoacetamide), sodium lauryl sarcosinate, sodium lauryl sulfate, sodium ether lauryl sulfate, and combinations thereof. In certain embodiments, the one or more surfactants is selected from zwitterionic surfactants. The one or more zwitterionic surfactants may be cocamidopropyl betaine. In further embodiments, the one or more surfactants is selected from sodium lauryl benzene sulfonate, dodecyl sodium sulfoacetate, N-2-ethyl laurate potassium sulfoacetamide), sodium lauryl sarcosinate, sodium lauryl sulfate, sodium ether lauryl sulfate, cocamidopropyl betaine, and combinations thereof. The one or more surfactants may be present in an amount from about 1% to about 5% by weight, about 1.5% to about 4.5% by weight, or about 2% to about 4.5% by weight, based on a total weight of the oral care composition.

In other embodiments, the invention is a method for oral care maintenance, comprising contacting the oral cavity of a subject in need thereof, with an oral care composition according to any one of the proceeding claims.

Further areas of applicability of the present disclosure will become apparent from the detailed description provided hereinafter. It should be understood that the detailed description and specific examples, while indicating the preferred embodiment of the disclosure, are intended for purposes of illustration only and are not intended to limit the scope of the disclosure.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other applications and methods. It is to be understood that the invention is not limited in its application to the details of any particular embodiment shown. The terminology used herein is for the purpose of description and not to limit the invention, its application, or uses.

As used herein and in the appended claims, the singular forms “a”, “an”, and “the” include plural references unless the context dictates otherwise. The singular form of any class of the ingredients refers not only to one chemical species within that class, but also to a mixture of those chemical species. The terms “a” (or “an”), “one or more” and “at least one” may be used interchangeably herein. The terms “comprising”, “including”, “containing”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range.

Unless otherwise specified, all percentages and amounts expressed herein and elsewhere in the specification should be understood to refer to percentages by weight of the total composition.

According to the present application, use of the term “about” in conjunction with a numeral value refers to a value that may be +/−5% of that numeral. As used herein, the term “substantially free” is intended to mean an amount less than about 5.0 weight %, less than 3.0 weight %, 1.0 wt. %; preferably less than about 0.5 wt. %, and more preferably less than about 0.25 wt. % of the composition.

Unless defined otherwise, all technical and scientific terms used herein have the same meanings as commonly understood by one of ordinary skill in the art to which this invention belongs. All patents, patent applications, publications, and other references cited or referred to herein are incorporated by reference in their entireties for all purposes. In the event of a conflict in a definition in the present disclosure and that of a cited reference, the present disclosure controls.

The present inventors have surprisingly and unexpectedly discovered that certain fatty acid salts may be used as thickening or gelling agents and are compatible with high water oral care compositions. Particularly, the present inventors have unexpectedly discovered that conventional thickening agents may be replaced with certain fatty acid salts to provide similar or comparable compatibility. Further, the present inventors have unexpectedly discovered that such compositions may use calcium carbonate abrasives at less than 30%.

As used herein, the term “high water” refers to an oral care composition, such as a toothpaste or oral gel, which comprises from 10% to 99% water, by weight of the composition. For example, the composition may comprise at least 10%, 15%, 20%, 25%, 30%, 35% or 40% water, up to a maximum of, for example, 60%, 70%, 80%, 90%, 95% or 99% water, by weight of the composition. As used herein, amounts of water refer to water added directly to the composition, as well as water added as part of ingredients or components which are added as aqueous solutions. In some embodiments, the composition comprises from 10% to 60% water, or from 10% to 50% water, or from 10% to 40% water, or from 20% to 50% water, or from 25% to 45% water, or from 30% to 40% water, or about 45% water, by weight of the composition.

In one aspect, the present disclosure therefore provides high water oral care compositions comprising an orally acceptable carrier, a thickening system, one or more abrasives, and one or more gelling agents, wherein the one or more gelling agents comprise a monovalent salt of a fatty acid.

Compositions disclosed herein may be or include an oral care composition. The oral care composition may be a high water oral care composition, such as a high water dentifrice or toothpaste. The oral care composition may include an orally acceptable vehicle, a thickening system, one or more abrasives, and one or more gelling agents. The gelling agents may be or include, but are not limited to, one or more fatty acid salts. In at least one implementation, the one or more fatty acid salts are monovalent salts of a fatty acid. In at least one implementation, the one or more fatty acid salts are divalent salts of a fatty acid. In at least one implementation, the one or more fatty acid salts are both a monovalent salt and a divalent salt of a fatty acid. In at least one implementation, the gelling agents are free, or substantially free, of fatty alcohols. As used herein, “substantially free of fatty alcohols” may refer to a composition that contains fatty alcohols in an amount of less than 5.0 weight %, less than 3.0 weight %, less than 1.0 weight %, less than 0.1 weight %, less than 0.05 weight %, less than 0.01 weight %, less than 0.005 weight %, or less than 0.0001 weight %, based on a total weight of the oral care composition. As used herein, “free of fatty alcohols” may refer to a composition that contains less than 0.5 weight %, less than 0.1 weight %, less than 0.05 weight %, less than 0.01 weight %, less than 0.005 weight %, or less than 0.0001 weight %, based on a total weight of the oral care composition.

It should be appreciated that all ingredients for use in the compositions described herein are orally acceptable. As used herein, the expression “orally acceptable” may define an ingredient that is present in a composition as described in an amount and form that does not render the composition unsafe for use in the oral cavity.

The oral care composition may be a single phase oral care product or single phase oral care composition. For example, all the components of the oral care composition may be maintained together with one another in a single phase and/or vessel. For example, all the components of the oral care composition may be maintained in a single phase, such as a single homogenous phase. The single homogenous phase may be an anhydrous product or an anhydrous composition.

The oral care composition may be or form at least a portion of one or more oral care products. The oral care composition may include or be combined with an orally acceptable vehicle to form the oral care product (e.g., the toothpaste). Illustrative oral care products may include, but are not limited to, a toothpaste (dentifrice), a prophylactic paste, a tooth polish, a tooth gel (e.g., a whitening gel), a chewing gum, a lozenge, a mouthwash, a whitening strip, a paint-on gel, varnish, veneer, and tube, syringe or dental tray comprising a gel or paste, or a gel or paste coated on an application support such as dental floss or a toothbrush (e.g., a manual, electric, sound, a combination thereof or ultrasound toothbrush). In one implementation, the oral care composition may be or may form at least a portion of a toothpaste.

In at least one implementation, the orally acceptable vehicle may be, or include, one or more of water, sorbitol, glycerin, propylene glycol, polyethylene glycol, block copolymers of ethylene oxide (EO) and propylene oxide (PO), and combinations thereof. In a preferred implementation, the orally acceptable vehicle may be, or include one or more, but is not limited to water, sorbitol, and glycerin. In certain embodiments, the orally acceptable vehicle is water and sorbitol. In other embodiments, the orally acceptable vehicle is water and glycerin. In other embodiments, the orally acceptable vehicle is water, sorbitol, and glycerin.

The orally acceptable vehicle may be present in an amount of from 10 weight % to about 80 weight %, based on a total weight of the oral care composition. For example, orally acceptable vehicle may be present in an amount of from about 10 weight %, about 15 weight %, or about 20 weight % to about 25 weight %, about 30 weight %, about 35 weight %, about 40 weight %, about 45 weight %, about 50 weight %, about 55 weight %, about 60 weight %, about 65 weight %, about 70 weight %, about 75 weight %, or about 80 weight %. In another example, the propylene glycol may be present in an amount of from about 10 weight % to about 75 weight %, about 20 weight % to about 75 weight %, about 30 weight % to about 75 weight %, about 40 weight % to about 75 weight %, about 50 weight % to about 75 weight %, about 50 weight % to about 70 weight %, or about 50 weight % to about 65 weight %, based on the total weight of the oral care composition. In an exemplary implementation, the orally acceptable vehicle may be present in an amount of about 50 weight % to about 75 weight %, preferably about 55 weight % to about 70 weight %, and more preferably about 60 weight % to about 70 weight %, based on the total weight of the oral care composition. In a preferred implementation, the propylene glycol may be present in an amount of about 55 weight % to about 70 weight %, or about 65 weight %.

In at least one implementation, the oral care product may include a thickening system having one or more silica thickeners. The one or more thickeners may be any orally acceptable thickener or thickening agent. Illustrative silica thickeners may be or include, but are not limited to, colloidal silica and fumed silica. Note that these silica thickeners are physically and functionally distinct from the particulate silica abrasives also present in the compositions, as the silica thickeners are very finely divided and provide little or no abrasive action. In one embodiment, the one or more silica thickeners comprises Zeodent 168, which may be purchased from Evonik in Essen, Germany. Other thickening agents are carboxyvinyl polymers, carrageenan, hydroxyethyl cellulose and water soluble salts of cellulose ethers such as sodium carboxymethyl cellulose and sodium carboxymethyl hydroxyethyl cellulose. Natural gums such as karaya, gum arabic, and gum tragacanth can also be incorporated. Colloidal magnesium aluminum silicate can also be used as component of the thickening composition to further improve the composition's texture. The thickening system may also include a cross-linked polyvinylpyrrolidone (PVP) polymer. The thickening system may also include POLYPLASDONE™ XL 10F, which is commercially available from Ashland Inc. of Covington, Ky. Additional illustrative thickeners may include, but are not limited to, carbomers (e.g., carboxyvinyl polymers), carrageenans (e.g., Irish moss, carrageenan, iota-carrageenan, etc.), high molecular weight polyethylene glycols (e.g., CARBOWAX®, which is commercially available from The Dow Chemical Company of Midland, Mich.), cellulosic polymers, hydroxyethylcellulose, carboxymethylcellulose, and salts thereof (e.g., CMC sodium), natural gums (e.g., karaya, xanthan, gum arabic, and tragacanth), colloidal magnesium aluminum silicate, hydrophilic polymers, such as carbomers, such as carboxymethylene polymers, such as acrylic acid polymers, and acrylic acid copolymers, and the like, and mixtures or combinations thereof. Carboxypolymethylene is a slightly acidic vinyl polymer with active carboxyl groups. One such carboxypolymethylene is CARBOPOL® 974 and/or 980, commercially available from Noveon, Inc. of Cleveland, Ohio. In at least one implementation, the one or more thickeners may be or include a cellulose ether, selected from one or more of hydroxyalkyl cellulose polymers, such as hydroxypropyl methyl cellulose (HPMC), hydroxypropyl cellulose, hyrdoxyethyl cellulose, methyl cellulose, ethylcellulose, carboxymethyl cellulose, and mixtures or combinations thereof.

In at least one implementation, the thickening system may include a single thickener. For example, the thickening system may include a colloidal silica or fumed silica. In another implementation, the thickening system may include a plurality of thickeners. For example, the thickening system may include a silica thickener and carrageenan. In another example, the thickening system may include a plurality of silica thickeners. In another example, the thickening system may include a silica thickener, sodium carboxymethylcellulose, and polyanionic cellulose.

The amount or concentration of the thickening system and/or the thickeners thereof present in the oral care product may vary. The amount of the thickening system and/or the thickeners thereof present in the oral care composition may be from about 0.5% to 10.0%, based on the total weight of the oral care composition. For example, the amount of the thickening system and/or the thickeners thereof present in the oral care composition may be from about 0.5 wt. %, about 1.0 wt. %, about 1.5 wt. %, about 2 wt. %, about 2.5 wt. %, about 3 wt. %, about 3.5 wt. %, about 4 wt. %, about 4.5 wt. %, about 5 wt. %, about 5.5 wt. %, or about 1 wt. % to about 5.5 wt. %, about 6 wt. %, about 6.5 wt. %, about 7 wt. %, about 7.5 wt. %, about 8 wt. %, about 8.5 wt. %, about 9 wt. %, or about 10 wt. %. In another example, the amount of the thickening system and/or the thickeners thereof present in the oral care composition thereof may be from about 0.5 wt. % to about 9 wt. %, about 1 wt. % to about 6 wt. %, about 1 wt. % to about 5.5 wt. %, about 1 wt. % to about 5 wt. %, about 1 wt. % to about 4.5 wt. %, about 2 wt. % to about 6 wt. %, about 2 wt. % to about 5.5 wt. %, about 2 wt. % to about 5 wt. %, or about 3 wt. % to about 6 wt. %. In a typical implementation, the amount of the thickening system and/or the thickeners thereof present in the oral care composition may be from about 3 wt. % to about 5.5 wt. %, more typically about 4.2 wt. %.

The oral care composition may include one or more gelling agents capable of or configured to thicken the oral care composition. Illustrative gelling agents may be or include, but are not limited to, one or more salts of a fatty acid. As used herein, the term “salt of a fatty acid” refers to an aliphatic monocarboxylic acid whose carboxylic acid functional group is in the form of a salt. The hydrocarbon chain of the fatty acid salt may be saturated or unsaturated (i.e., alkyl, alkenyl or alkynyl hydrocarbon chains). In addition, the hydrocarbon chain may be straight or branched. Moreover, in some embodiments, hydrogens in the hydrocarbon chain may be substituted. In preferred embodiments, the fatty acid is a C₁₂-C₃₂ fatty acid.

The salt of fatty acid may be or include an unsaturated linear and/or a saturated linear fatty acid. In at least one implementation, the fatty acid may be or include one or more unsaturated linear or saturated linear C₁₂-C₃₂ fatty acids. It should be appreciated that the oral care composition may include any one or more fatty acids within the indicated carbon number range. For example, the gelling agents in the oral care composition may be or include salts of lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, linoleic acid, arachidonic acid, palmitoleic acid, oleic acid, and the like, and mixtures or combinations thereof. In an exemplary implementation the oral care composition includes salts of palmitic acid and/or stearic acid as gelling agents.

Unsaturated fatty acids useful in the invention may be almost, but not fully hydrogenated. The amount of hydrogenation may be measured by determining the iodine value. The iodine value can be measured by ASTM D5554-95 (2006). In certain embodiments, the unsaturated fatty acid is a C₁₂-C₃₂ fatty acid. In certain embodiments, unsaturated fatty acid has an iodine value of less than 20. In certain embodiments, unsaturated fatty acid has an iodine value of less than 10. In certain embodiments, unsaturated fatty acid has an iodine value of less than 5. In certain embodiments, unsaturated fatty acid has an iodine value of less than 1. In further embodiments, the unsaturated fatty acid is a C₁₆₋₁₈ fatty acid and has an iodine value of less than 20, 10, 5, or less than 1.

Embodiments of the invention utilize the salt of fatty acid, wherein the salt comprises a monovalent salt, a divalent salt, or a combination thereof. In preferred embodiments, the salt of fatty acid comprises a sodium salt. In other embodiments, the salt of fatty acid comprises a potassium salt. In other embodiments, the salt of fatty acid comprises a lithium salt. In other embodiments, the salt of fatty acid comprises salt selected from sodium salt, potassium salt, lithium salt, and a combination of two or more thereof. In other embodiments, the salt of fatty acid is a combination of sodium and potassium salt. In certain embodiments, the salt of fatty acid comprises sodium palmitate. In certain embodiments, the salt of fatty acid comprises sodium stearate. In certain embodiments, the salt of fatty acid comprises potassium palmitate. In certain embodiments, the salt of fatty acid comprises potassium stearate. In certain embodiments, the salt of fatty acid is selected from sodium palmitate, sodium stearate, potassium palmitate, potassium stearate, and a combination of two or more thereof.

Embodiments of the invention may utilize the divalent salt of fatty acid. In preferred embodiments, the divalent salt of fatty acid comprises a zinc salt. In other embodiments, the divalent salt of fatty acid comprises a magnesium salt. In other embodiments, the divalent salt of fatty acid comprises a calcium salt. In other embodiments, the divalent salt of fatty acid is selected from zinc salt, magnesium salt, calcium salt, and a combination of two or more thereof. In other embodiments, the divalent salt of fatty acid is a combination of zinc and calcium salt. In certain embodiments, the divalent salt of fatty acid comprises zinc palmitate. In certain embodiments, the divalent salt of fatty acid comprises zinc stearate. In certain embodiments, the divalent salt of fatty acid comprises calcium palmitate. In certain embodiments, the divalent salt of fatty acid comprises calcium stearate. In certain embodiments, the divalent salt of fatty acid comprises magnesium palmitate. In certain embodiments, the divalent salt of fatty acid comprises magnesium stearate. In certain embodiments, the divalent salt of fatty acid is selected from zinc palmitate, zinc stearate, calcium palmitate, calcium stearate, magnesium palmitate, magnesium stearate, and a combination of two or more thereof.

Embodiments of the invention utilize the salt of fatty acid, wherein the salt comprises both a monovalent salt of a fatty acid and a divalent salt of a fatty acid. In preferred embodiments, the salt comprises a sodium salt and a calcium salt. In other embodiments, the salt comprises a sodium salt and a zinc salt. In certain embodiments, the salt comprises a sodium salt and a calcium salt and a zinc salt. In further embodiments, the salt comprises a potassium salt and a calcium salt. In other embodiments, the salt comprises a potassium salt and a zinc salt. In certain embodiments, the salt comprises a potassium salt and a calcium salt and a zinc salt.

The amount or concentration of the gelling agent(s) may vary widely. In at least one example, the gelling agent(s) may be present in an amount greater than or equal to 0.1 weight % and less than or equal to 10 weight %, based on the total weight of the oral care composition. For example, the gelling agents may be present in an amount of from about 0.1 weight %, about 0.2 weight %, about 0.4 weight %, about 0.6 weight %, about 0.8 weight %, about 1 weight %, about 1.5 weight %, about 2 weight %, about 2.5 weight %, or about 3 weight % to about 3.5 weight %, about 4 weight %, about 4.5 weight %, about 5 weight %, about 5.5 weight %, about 6.0 weight %, about 6.5 weight %, about 7.0 weight %, about 7.5 weight %, about 8.0 weight %, about 8.5 weight %, about 9.0 weight %, about 9.5 weight %, or about 10.0 weight %, based on the total weight of the oral care composition. In another example, the gelling agents may be present in an amount of from about 1 weight % to about 10 weight %, about 1.5 weight % to about 9.5 weight %, about 2 weight % to about 8 weight %, about 2.5 weight % to about 7.5 weight %, or about 3 weight % to about 7 weight %. In another example, the gelling agents may be present in an amount of from about 1 weight %, about 2 weight %, about 3 weight %, about 4 weight %, about 5 weight %, about 6 weight %, about 7 weight %, about 8 weight %, about 9 weight %, or about 10 weight % to about 10 weight %, about 20 weight %, about 25 weight %, about 30 weight %, about 35 weight %, about 40 weight %, about 45 weight %, or about 50 weight %, based on the total weight of the oral care composition. In a preferred embodiment, the one or more gelling agents may be present in an amount of from about 1 weight % to about 10 weight %, about 2 weight % to about 8 weight %, about 3 weight % to about 7 weight %, based on the total weight of the oral care composition.

The compositions of the present invention may optionally comprise additional ingredients suitable for use in oral care compositions. Examples of such ingredients include active agents, such as a fluoride source and/or a phosphate source. The compositions may be formulated in a suitable dentifrice base, e.g., comprising abrasives, e.g., silica abrasives, surfactants, foaming agents, vitamins, polymers, enzymes, humectants, thickeners, additional antimicrobial agents, preservatives, flavorings, colorings, and/or combinations thereof. Examples of suitable dentifrice bases are known in the art. Alternatively, the compositions may be formulated as a gel (e.g., for use in a tray), chewing gum, lozenge or mint. Examples of suitable additional ingredients that can be employed in the compositions of the present disclosure are discussed in more detail below.

Active Agents: The compositions of the disclosure may comprise various other agents that are active to protect and enhance the strength and integrity of the enamel and tooth structure and/or to reduce bacteria and associated tooth decay and/or gum disease or to provide other desired benefits. Effective concentration of the active ingredients used herein will depend on the particular agent and the delivery system used. The concentration will also depend on the exact salt or polymer selected. For example, where the active agent is provided in salt form, the counterion will affect the weight of the salt, so that if the counterion is heavier, more salt by weight will be required to provide the same concentration of active ion in the final product.

Compositions of the disclosure may contain from 0.1 to 1 wt. % of an antibacterial agent, such as about 0.3 wt. %. Any suitable antimicrobial actives can be employed.

Fluoride Ion Source: The oral care compositions can include one or more additional fluoride ion sources, e.g., soluble fluoride salts. A wide variety of fluoride ion-yielding materials can be employed as sources of soluble fluoride in the present compositions. Examples of suitable fluoride ion-yielding materials are found in U.S. Pat. No. 3,535,421, to Briner et al.; U.S. Pat. No. 4,885,155, to Parran, Jr. et al. and U.S. Pat. No. 3,678,154, to Widder et al, the disclosure of each of which is hereby incorporated by reference in their entirety. Representative fluoride ion sources include, but are not limited to, sodium fluoride, potassium fluoride, sodium monofluorophosphate, sodium fluorosilicate, ammonium fluorosilicate, amine fluoride, ammonium fluoride, and combinations thereof. In certain embodiments the fluoride ion source includes sodium fluoride, sodium monofluorophosphate as well as mixtures thereof. In certain embodiments, the oral care composition of the disclosure may contain stannous fluoride and any additional source of fluoride ions or fluorine-providing agents in amounts sufficient to supply, in total, from 25 ppm to 25,000 ppm (mass fraction) of fluoride ions, generally at least 500 ppm, e.g., from 500 to 2000 ppm, e.g., from 1000 to 1600 ppm, e.g., about 1450 ppm. The appropriate level of fluoride will depend on the particular application. A toothpaste for general consumer use would typically have from 1000 to about 1500 ppm, with pediatric toothpaste having somewhat less. A dentifrice or coating for professional application could have as much as 5,000 or even about 25,000 ppm fluoride. Additional fluoride ion sources may be added to the compositions of the disclosure at a level of from 0.01 wt. % to 10 wt. % in one embodiment or from 0.03 wt. % to 5 wt. %, and in another embodiment from 0.1 wt. % to 1 wt. % by weight of the composition. As discussed above, weights of fluoride salts to provide the appropriate level of fluoride ion will vary based on the weight of the counterion in the salt.

Abrasives: The compositions of the disclosure include one or more abrasives. Examples of suitable abrasives include, but are not limited to, calcium salt, for example, a calcium phosphate abrasive, e.g., tricalcium phosphate (Ca₃(PO₄)₂), hydroxyapatite (Ca₁₀(PO₄)₆(OH)₂), dicalcium phosphate dihydrate (CaHPO₄·2H₂O, also sometimes referred to herein as DiCal), anhydrous dicalcium phosphate, calcium pyrophosphate, or calcium carbonate salts. Calcium carbonate salts suitable for use include natural calcium carbonate (NCC), precipitated calcium carbonate (PCC), and mixtures thereof. Further suitable abrasives which may be included in the compositions include, but are not limited to silica abrasives, aluminum oxide, aluminum silicate, calcined alumina, bentonite, other siliceous materials, insoluble phosphates, natural calcium carbonate (NCC), precipitated calcium carbonate (PCC), and mixtures thereof. In some embodiments, the one or more abrasives comprises calcium carbonate abrasive. In some embodiments, the one or more abrasives is selected from precipitated calcium carbonate (PCC), natural calcium carbonate (NCC), and combinations thereof. In some embodiments, the one or more abrasives is selected from sodium metaphosphate, potassium metaphosphate, aluminum silicate, calcined alumina, bentonite, other siliceous materials, and combinations thereof. In certain preferred embodiments, the one or more abrasive is selected from natural calcium carbonate (NCC), precipitated calcium carbonate (PCC), dicalcium phosphate dihydrate, and combinations thereof. Examples of silica abrasives include, but are not limited to, precipitated or hydrated silicas having a mean particle size of up to about 20 microns (such as Zeodent 105 and Zeodent 114 marketed by J.M. Huber Chemicals Division, Havre de Grace, Md. 21078); Sylodent 783 (marketed by Davison Chemical Division of W.R. Grace & Company); or Sorbosil AC 43 (from PQ Corporation).

Abrasive polishing materials useful herein, as well as the other abrasives, generally have an average particle size ranging between 0.1 and 30 microns, such as between 5 and 15 microns. The silica abrasives can be from precipitated silica or silica gels, such as the silica xerogels described in U.S. Pat. No. 3,538,230, to Pader et al. and U.S. Pat. No. 3,862,307, to Digiulio, the disclosures of which are incorporated herein by reference in their entireties. Particular silica xerogels are marketed under the trade name Syloid® by the W. R. Grace & Co., Davison Chemical Division. The precipitated silica materials include those marketed by the J. M. Huber Corp. under the trade name Zeodent®, including the silica carrying the designation Zeodent 115 and 119. These silica abrasives are described in U.S. Pat. No. 4,340,583, to Wason, the disclosure of which is incorporated herein by reference in its entirety. In certain embodiments, abrasive materials useful in the practice of the oral care compositions in accordance with the disclosure include silica gels and precipitated amorphous silica having an oil absorption value of less than 100 cc/100 g silica, such as from 45 cc/100 g to 70 cc/100 g silica. Oil absorption values are measured using the ASTA Rub-Out Method D281. In certain embodiments, the silicas are colloidal particles having an average particle size of from 3 microns to 12 microns, and from 5 to 10 microns. Examples of low oil absorption silica abrasives useful in the practice of the disclosure are marketed under the trade designation Sylodent XWA® by Davison Chemical Division of W.R. Grace & Co., Baltimore, Md. 21203. Sylodent 650 XWA®, a silica hydrogel composed of particles of colloidal silica having a water content of 29% by weight averaging from 7 to 10 microns in diameter, and an oil absorption of less than 70 cc/100 g of silica is an example of a low oil absorption silica abrasive useful in the practice of the present disclosure.

In certain embodiments, the composition uses one or more abrasives. In certain embodiments, the one or more abrasives comprise calcium salt, silica, or sodium metaphosphate. In certain embodiments, the calcium salt is selected from tricalcium phosphate, hydroxyapatite, dicalcium phosphate dihydrate, anhydrous dicalcium phosphate, calcium pyrophosphate, natural calcium carbonate, precipitated calcium carbonate, and mixtures thereof.

Any suitable amount of abrasive can be employed. Examples of suitable amounts include 10 wt. % or more dry weight of particles, such as from 15 wt. % to 30 wt. %, from 15 wt. % to 25 wt. %, or about 20 wt. %, based on the total weight of the composition.

Foaming agents: The oral care compositions of the disclosure also may include an agent to increase the amount of foam that is produced when the oral cavity is brushed. Illustrative examples of agents that increase the amount of foam include, but are not limited to polyoxyethylene and certain polymers including, but not limited to, alginate polymers. The polyoxyethylene may increase the amount of foam and the thickness of the foam generated by the oral care compositions of the present disclosure. Polyoxyethylene is also commonly known as polyethylene glycol (“PEG”) or polyethylene oxide. The polyoxyethylenes suitable for compositions of the present disclosure may have a molecular weight of from 200,000 to 7,000,000. In one embodiment the molecular weight may be from 600,000 to 2,000,000 and in another embodiment from 800,000 to 1,000,000. Polyox™ is the trade name for the high molecular weight polyoxyethylene produced by Union Carbide. The foaming agent, (e.g., polyoxyethylene) may be present in an amount of from 0.1% to 50%, in one embodiment from 0.5% to 20% and in another embodiment from 1% to 10%, or from 2% to 5% by weight of the oral care composition.

Surfactants: The compositions useful in the compositions of the present disclosure may contain anionic surfactants. In certain embodiments, the anionic surfactant may be water-soluble salts of higher fatty acid monoglyceride monosulfates, such as the sodium salt of the monosulfated monoglyceride of hydrogenated coconut oil fatty acids such as sodium N-methyl N-cocoyl taurate, sodium cocomonoglyceride sulfate. In further embodiments, the anionic surfactant may be higher alkyl sulfates, such as sodium lauryl sulfate. In still further embodiments, the anionic surfactant may be higher alkyl-ether sulfates, e.g., of formula CH₃(CH₂)ₘCH₂(OCH₂CH₂)ₙOSO₃X, wherein m is 6-16, e.g., 10, n is 1-6, e.g., 2, 3 or 4, and X is Na or K; for example sodium laureth-2 sulfate (CH₃(CH₂)₁₀CH₂(OCH₂CH₂)₂OSO₃Na). In other embodiments, the anionic surfactant may be higher alkyl aryl sulfonates such as sodium dodecyl benzene sulfonate (sodium lauryl benzene sulfonate). In still further embodiments, the anionic surfactant may be higher alkyl sulfoacetates, such as sodium lauryl sulfoacetate (dodecyl sodium sulfoacetate), higher fatty acid esters of 1,2 dihydroxy propane sulfonate, sulfocolaurate (N-2-ethyl laurate potassium sulfoacetamide) and sodium lauryl sarcosinate. By “higher alkyl” is meant, e.g., C₆₋₃₀ alkyl. In certain embodiments, the anionic surfactants useful herein include the water-soluble salts of alkyl sulfates having from 10 to 18 carbon atoms in the alkyl radical and the water-soluble salts of sulfonated monoglycerides of fatty acids having from 10 to 18 carbon atoms. Sodium lauryl sulfate, sodium lauroyl sarcosinate and sodium coconut monoglyceride sulfonates are examples of anionic surfactants of this type. In particular embodiments, the anionic surfactant is selected from sodium lauryl sulfate and sodium ether lauryl sulfate. In a particular embodiment, the compositions of the disclosure comprise sodium lauryl sulfate. The anionic surfactant may be present in an amount which is effective, e.g., >0.01% by weight of the formulation, but not at a concentration which would be irritating to the oral tissue, e.g., <10%, and optimal concentrations depend on the particular formulation and the particular surfactant. In one embodiment, the anionic surfactant is present in a toothpaste at from 0.3% to 4.5% by weight, e.g., about 1.5%. The compositions of the disclosure may optionally contain mixtures of surfactants, e.g., comprising anionic surfactants and other surfactants that may be anionic, cationic, zwitterionic or nonionic. Generally, suitable surfactants are those which are reasonably stable throughout a wide pH range. Surfactants are described more fully, for example, in U.S. Pat. No. 3,959,458, to Agricola et al.; U.S. Pat. No. 3,937,807, to Haefele; and U.S. Pat. No. 4,051,234, to Gieske et al, the disclosures of which are incorporated herein by reference in their entireties.

The inventive compositions of the present disclosure may contain zwitterionic surfactants. In certain embodiments, the zwitterionic surfactant is cocamidopropyl betaine. In certain embodiments, the inventive compositions may contain an amphoteric surfactant. The amphoteric and zwitterionic surfactants may be or include, but are not limited to, derivatives of C₈₋₂₀ aliphatic secondary and tertiary amines having an anionic group such as carboxylate, sulfate, sulfonate, phosphate or phosphonate. Illustrative amphoteric and zwitterionic surfactants may include, but are not limited to, sultaines and betaines, such as cocamidopropyl betaine (CAPB), derivatives of aliphatic secondary and tertiary amines in which the aliphatic radical can be a straight or branched chain and wherein one of the aliphatic substituents contains about 8-18 carbon atoms and one contains an anionic water-solubilizing group, such as carboxylate, sulfonate, sulfate, phosphate or phosphonate, or the like, and combinations thereof. In at least one preferred implementation, the surfactant of the oral care composition includes sodium lauryl sulfate and at least one amphoteric and/or zwitterionic surfactant. For example, the oral care composition includes one or more sodium lauryl sulfate and one or more betaines, such as cocamidopropyl betaine.

The surfactants may be or include, but are not limited to, one or more anionic surfactants, one or more amphoteric surfactant, one or more zwitterionic surfactants, or combinations thereof.

The one or more surfactants can be present in the compositions of the present disclosure in from 0.1% to 5.0%, in another embodiment from 0.3% to 3.0% and in another embodiment from 0.5% to 2.0% by weight of the total composition. In other embodiments, the one or more surfactants are present in an amount from about 1% to about 5% by weight, about 1.5% to about 4.5% by weight, or about 2% to about 4.5% by weight, based on a total weight of the oral care composition.

In some embodiments, the compositions of the present disclosure include a zwitterionic surfactant, for example a betaine surfactant, for example cocamidopropyl betaine, e.g. in an amount of from 0.1% to 4.5% by weight, e.g. from 0.5 to 2% by weight cocamidopropyl betaine.

Tartar control agents: In various embodiments of the present disclosure, the compositions comprise an anticalculus (tartar control) agent. Suitable anticalculus agents include, without limitation, phosphates and polyphosphates (for example pyrophosphates and tripolyphosphates), polyaminopropanesulfonic acid (AMPS), hexametaphosphate salts, zinc citrate trihydrate, polypeptides, polyolefin sulfonates, polyolefin phosphates, and diphosphonates. The compositions of the disclosure thus may comprise phosphate salts in addition to the zinc phosphate. In particular embodiments, these salts are alkali phosphate salts, e.g., salts of alkali metal hydroxides or alkaline earth hydroxides, for example, sodium, potassium or calcium salts. “Phosphate” as used herein encompasses orally acceptable mono- and polyphosphates, for example, P.sub.1-6 phosphates, for example monomeric phosphates such as monobasic, dibasic or tribasic phosphate; and dimeric phosphates such as pyrophosphates; and multimeric phosphates, such as tripolyphosphates, tetraphosphates, hexaphosphates and hexametaphosphates (e.g., sodium hexametaphosphate). In particular examples, the selected phosphate is selected from alkali dibasic phosphate and alkali pyrophosphate salts, e.g., selected from sodium phosphate dibasic, potassium phosphate dibasic, dicalcium phosphate dihydrate, calcium pyrophosphate, tetrasodium pyrophosphate, tetrapotassium pyrophosphate, sodium tripolyphosphate, and mixtures of any of two or more of these. In a particular embodiment, for example the compositions may comprise tetrasodium pyrophosphate in an amount of from 0.5 to 5% by weight, e.g., 1-3%, or 1-2% or about 2% by weight of the composition. In another embodiment, the compositions may comprise a mixture of tetrasodium pyrophosphate (TSPP) and sodium tripolyphosphate (STPP), e.g., in proportions of TSPP at from 0.5 to 5 wt. %, such as from 1 to 2 wt. % and STPP at from 0.5% to 6 wt. %, such as 1 to 4%, or 2 to 3% by weight of the composition. Such phosphates are provided in an amount effective to reduce erosion of the enamel, to aid in cleaning the teeth, and/or to reduce tartar buildup on the teeth, for example in an amount of from 0.2 to 20 wt. %, e.g., from 1 to 15 wt. %, by weight of the composition.

Flavoring Agents: The oral care compositions of the disclosure may also include a flavoring agent. Flavoring agents which are used in the practice of the present disclosure include, but are not limited to, essential oils as well as various flavoring aldehydes, esters, alcohols, and similar materials. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Certain embodiments employ the oils of peppermint and spearmint. The flavoring agent may be incorporated in the oral composition at a concentration of from 0.1 to 5% by weight e.g., from 0.5 to 1.5% by weight.

Other Polymers: The oral care compositions of the disclosure may also include additional polymers to adjust the viscosity of the formulation or enhance the solubility of other ingredients. Such additional polymers include polyethylene glycols, polysaccharides (e.g., cellulose derivatives, for example carboxymethyl cellulose, hydroxymethyl cellulose, ethyl cellulose, microcrystalline cellulose or polysaccharide gums, for example xanthan gum, guar gum or carrageenan). Acidic polymers, for example polyacrylate gels, may be provided in the form of their free acids or partially or fully neutralized water soluble alkali metal (e.g., potassium and sodium) or ammonium salts. In one embodiment, the oral care composition may contain PVP. PVP generally refers to a polymer containing vinylpyrrolidone (also referred to as N-vinylpyrrolidone, N-vinyl-2-pyrrolidione and N-vinyl-2-pyrrolidinone) as a monomeric unit. The monomeric unit consists of a polar imide group, four non-polar methylene groups and a non-polar methane group. Other polymers among those useful herein include polyvinylpyrrolidone (PVP), cross-linked polyvinyl pyrrolidone, polyvinylpyrrolidone-vinyl acetate (PVP-VA) copolymer, polyvinyl alcohol, polyacrylic acid, poly acrylate polymer, cross-linked polyacrylate polymer, cross-linked polyacrylic acid (such as Carbopol™), polyethylene oxide, polyethylene glycol, poly vinyl alkyl ether-maleic acid copolymer (such as Gantrez™) and carboxy vinyl polymer; natural gums such as sodium alginate, carrageenan, xantham gum, gum acacia, arabic gum, guar gum, pullulan, agar, chitin, chitosan, pectin, karaya gum, zein, hordein, gliadin, locust bean gum, tragacantha and other polysaccharides; starches such as maltodextrin, amylose, high amylose starch, corn starch, potato starch, rice starch, tapioca starch, pea starch, sweet potato starch, barley starch, wheat starch, waxy corn starch, modified starch (such as hydroxypropylated high amylose starch), dextrin, levan, elsinan and gluten; and proteins such as collagen, whey protein isolate, casein, milk protein, soy protein and gelatin. In certain embodiments, the solid composition disclosed herein may be free of water-soluble polymers, such as free of PVP and PVP-VA.

In some embodiments, the compositions of the disclosure comprise one or more polyethylene glycols, for example, polyethylene glycols in a molecular weight range from 200 to 800. For example, the compositions may comprise one or more of polyethylene glycol 200, polyethylene glycol 300, polyethylene glycol 400, polyethylene glycol, 600 or polyethylene glycol 800.

Humectants: Within certain embodiments of the oral compositions, it is also desirable to incorporate a humectant to prevent the composition from hardening upon exposure to air. Certain humectants can also impart desirable sweetness or flavor to dentifrice compositions. Suitable humectants include edible polyhydric alcohols such as glycerin, sorbitol, xylitol, propylene glycol as well as other polyols and mixtures of these humectants. In one embodiment of the disclosure, the principal humectant is one of glycerin, sorbitol or a combination thereof. The humectant may be present at levels of greater than 15 wt. %, such as from 15 wt. % to 55 wt. %, or from 20 wt. % to 50 wt. %, or from 20 wt. % to 40 wt. %, or about 20% or about 30% or about 40%, based on the total weight of the composition.

Other optional ingredients: In addition to the above-described components, the embodiments of this disclosure can contain a variety of optional oral care ingredients some of which are described below. Optional ingredients include, for example, but are not limited to, adhesives, sudsing agents, flavoring agents, sweetening agents such as sodium saccharin, additional antiplaque agents, abrasives, aesthetics such as TiO.sub.2 coated mica or other coloring agents, such as dyes and/or pigments.

In some embodiments, the compositions of the present disclosure can have any pH suitable for in a product for use in oral care. Examples of suitable pH ranges are from 5 to 10.5, such as from about 5.5 to about 10, about 6 to about 10, about 6 to about 9, about 6.5 to about 8, or about 6.5 to about 7.5, or about 7.0.

In another aspect, the present disclosure provides a method of treatment or prevention of erosive tooth demineralization, gingivitis, plaque, and/or dental caries, the method comprising the application to the oral cavity of a person in need thereof a composition according to the invention described herein, e.g., by brushing, for example, one or more times per day.

In another aspect, the present disclosure provides a method of oral care maintenance, comprising contacting the oral cavity of a subject in need thereof, with a composition described herein (e.g., any of compositions described herein). The methods comprise applying any of the compositions as described herein to the teeth, e.g., by brushing, or otherwise administering the compositions to the oral cavity of a subject in need thereof. The compositions can be administered regularly, such as, for example, one or more times per day. In various embodiments, administering the compositions of the present disclosure to a patient can provide one or more of the following benefits: (i) reduce hypersensitivity of the teeth, (ii) reduce plaque accumulation, (iii) reduce or inhibit demineralization and promote remineralization of the teeth, (iv) inhibit microbial biofilm formation in the oral cavity, (v) reduce or inhibit gingivitis, (vi) promote healing of sores or cuts in the mouth, (vii) reduce levels of acid producing bacteria, (viii) increase relative levels of non-cariogenic and/or non-plaque forming bacteria, (ix) reduce or inhibit formation of dental caries, (x) reduce, repair or inhibit pre-carious lesions of the enamel, e.g., as detected by quantitative light-induced fluorescence (QLF) or electrical caries measurement (ECM), (xi) treat, relieve or reduce dry mouth, (xii) clean the teeth and oral cavity, (xiii) reduce erosion, (xiv) whiten teeth; (xv) reduce tartar build-up, and/or (xvi) promote systemic health, including cardiovascular health, e.g., by reducing potential for systemic infection via the oral tissues. Further embodiments provide methods wherein at least one tooth is remineralized after administration of a composition as described herein.

The present application further discloses a method of making any of the compositions of the present disclosure. The method comprises combining an orally acceptable vehicle with a thickening system, one or more abrasives, and one or more gelling agents, wherein the one or more gelling agents comprise a monovalent salt of a fatty acid, to form a high water oral care composition. The amount of water employed in the mixture can be any of the amounts recited herein for the compositions of the present disclosure. Any standard mixing techniques can be employed to combine the ingredients and form a stable composition.

In another aspect, the present disclosure provides a method of treatment or prevention of erosive tooth demineralization, gingivitis, plaque, and/or dental caries, the method comprising the application to the oral cavity of a person in need thereof a composition according to the invention (e.g., composition or methods claimed and described herein), e.g., by brushing, for example, one or more times per day.

### EXAMPLES

The examples and other implementations described herein are exemplary and not intended to be limiting in describing the full scope of compositions and methods of this disclosure. Equivalent changes, modifications and variations of specific implementations, materials, compositions and methods may be made within the scope of the present disclosure, with substantially similar results.

### Example 1

An oral care composition was prepared by combining the ingredients/components according to Table 1 to make Sample 1. For this composition, NCC was used as the abrasive and along with an anionic surfactant.

TABLE 1
Composition for Sample 1

| Material | Quantity (wt. % of total composition) |
|---|---|
| Sorbitol | 33 |
| Water | 34.72 |
| Sodium stearate | 4 |
| Thickening silica | 3.5 |
| Sodium monofluorophosphate | 0.76 |
| Sweetener | 0.27 |
| Abrasive | 20 |
| Flavor | 0.95 |
| Carrageenan | 0.8 |
| Surfactant | 2 |

The composition of Sample 1 formed a solid paste. The initial Brookfield viscosity was 299,849 cPs. The 10% pH in water was 9.98. The toothpaste remained as a non-flowable solid paste in a glass jar during aging at 40° C. and 49° C. for 3 months without showing any phase separation.

To test the chelating function of the composition, analysis was performed whereby CaCl₂·2H₂0 was added to a 1:1 toothpaste water slurry. The results are shown in Table 2 (below).

TABLE 2
Ca²⁺-Chelating test for stearate toothpaste

| | | Added | Added | Detected | Slurry |
|---|---|---|---|---|---|
| Water | Toothpaste | CaCl₂•2H₂O, | Ca²⁺ | Ca²⁺, | state of |
| (gram) | (gram) | (gram) | (ppm) | (ppm) | matter |
| 25.3220 | 25.0018 | 0 | 0 | 550 | liquid |
| 25.6157 | 25.0588 | 0.0538 | 289 | 583 | liquid |
| 25.0852 | 25.2551 | 0.1051 | 567 | 619 | liquid |
| 25.1260 | 25.0526 | 0.2043 | 1103 | 585 | liquid |
| 25.3410 | 25.1487 | 0.3106 | 1664 | 769 | solid |
| 25.3982 | 25.0243 | 0.4008 | 2146 | 957 | solid |
| 25.2515 | 25.2763 | 0.5075 | 2706 | 1359 | solid |

The data described in Table 2 (above) shows that detection of added Ca²⁺ concentration was significantly reduced compared to the amount added. Theoretically, for toothpaste without Ca²⁺ chelating power, the added Ca²⁺ should be equal to the detected Ca²⁺. Therefore, the data described in Table 2 (above) demonstrates that the calcium chelating capabilities of exemplary compositions of the present invention.

### Example 2

Oral care compositions were prepared by combining the ingredients/components according to Table 3. For these compositions, PCC was used as the abrasive and an anionic surfactant.

TABLE 3
Composition for Sample 2 and Sample 3

| | Quantity (wt. % of total composition) | |
|---|---|---|
| Material | Sample 2 | Sample 3 |
| Sorbitol | 23 | 20 |
| Water | 23.826 | 45.5689 |
| Sodium carboxymethyl cellulose | 0.9 | 1 |
| Sodium bicarbonate | 0.5 | 3 |
| Sodium monofluorophosphate | 1.1 | 1.1 |
| Sweetener | 0.2 | 0.2 |
| Abrasive | 44 | 22 |
| Surfactant | 4.59 | 1.3311 |
| Tetrasodium pyrophosphate | 0.5 | |
| Dentifrice | 1 | 0.8 |
| Preservative | 0.3 | |
| Sodium hydroxide 50% | 0.084 | |
| Sodium stearate | | 2 |
| Thickening silica | | 3 |

Both Sample 2 and Sample 3 formed a solid phase and remained within a non-flowable solid phase in glass jars under 40° C. and 49° C. aging conditions without phase separation. Their specific gravities are 1.533 and 1.277, respectively. Their Brookfield viscosities were about 591,694 cPs and 309,799 cPs, respectively, which represents that the toothpastes have good stand-up and stability. Phase separation instability indices were measured by a high-speed centrifugation predictive method and resulted in 0.064 and 0.093, respectively. Thus, their instability indices were very close (the lower close to 0, the better) and close to zero phase separation would be expected to occur for 3 years.

### Example 3

To characterize optimal sodium stearate levels, four formulae were prepared having either 4%, 2%, 1%, or 0% sodium stearate. The results are listed in Table 4.

TABLE 4
Aging results for different level of sodium stearate

| | Sodium | Brookfield | | | |
|---|---|---|---|---|---|
| Sample # | Stearate | Viscosity (cPs) | RT | 40° C. | 49° C. |
| 4 | 4% | hard to squeeze | | | |
| | | (like a rock) | | | |
| 5 | 2% | 309,799 | non- | non- | non- |
| | | | flowable | flowable | flowable |
| 6 | 1% | 121,047 | barely | flowable | flowable |
| | | | flowable | | |
| 7 | 0% | 73,397 | Flowable | flowable | flowable |

RT = room temperature

### Example 4

An oral care composition was prepared by combining the ingredients/components according to Table 5 (below) to make Sample 8. In this composition, dicalcium phosphate dihydrate (DiCal) was used as the abrasive along with an anionic surfactant.

TABLE 5
Composition of Sample 8

| Ingredient | Quantity (wt. % of total composition) |
|---|---|
| Glycerin | 29.57 |
| Water | 34.24 |
| Sodium stearate | 4 |
| Thickening silica | 3.5 |
| Sodium monofluorophosphate | 0.76 |
| Sweetener | 0.18 |
| Abrasive | 24 |
| Flavor | 0.95 |
| Sodium carboxymethyl cellulose | 1.3 |
| and polyanionic cellulose | |
| Surfactant | 1.5 |

The composition of Sample 8 was initially and remained a non-flowable solid paste in glass jars at 40° C. and 49° C. after 3 months of aging without phase separation. The initial Brookfield viscosity was 371,499 cPs. The 10% pH in water was 7.82.

### Example 5

A whitening oral care composition was prepared by combining the ingredients/components according to Table 6 to make Sample 9. In this composition, high cleaning silica was used as the abrasive along with both a zwitterionic surfactant and an anionic surfactant.

TABLE 6
Composition of Sample 9

| Material | Quantity (wt. % of total composition) |
|---|---|
| Sorbitol | 22.377 |
| Water | 33.2 |
| Glycerin | 10 |
| Sodium stearate | 0-4 |
| Thickening silica | 3.5 |
| Sodium fluoride | 0.243 |
| Sweetener | 0.03 |
| Abrasive | 20 |
| Flavor | 1.3 |
| Sodium carboxymethyl cellulose | 1.3 |
| (CMC) | |
| Surfactant | 4.25 |
| Xantham gum | 0.3 |

The composition of Sample 9 was evaluated for whitening effects based on sodium stearate concentrations. Table 7 shows the results of the analysis.

TABLE 7
Effect of sodium stearate on Brookfield
viscosity and L*a*b* numbers.

| | Sodium | | | | |
|---|---|---|---|---|---|
| Sample # | Stearate | Brookfield | L | a | b |
| 10 | 5% | 550,504 | 84.9 | −1.6 | 3.3 |
| 11 | 4% | 296,259 | 84.1 | −1.1 | 3.7 |
| 12 | 3% | 235,725 | 82.7 | −1.6 | 3.3 |
| 13 | 2% | 140,409 | 73.2 | −1.6 | 1.2 |

Table 7 shows that by changing the sodium stearate level from 2% to 5% while keeping the CMC gum at 0.8% and xanthan gum at 0.3%, the Brookfield viscosity increases from 140,000 cPs to 550,000 cPs. The optimal squeezable pressure was at 4% sodium stearate (Sample 11). In addition, determination of the L*a*b* numbers for these formulas were also performed. Accordingly, the data described in Table 7 (above), highlight the impact that sodium stearate has on the viscosity and whitening benefits provided by the claimed invention.

While the present invention has been described with reference to several embodiments, which embodiments have been set forth in considerable detail for the purposes of making a complete disclosure of the invention, such embodiments are merely exemplary and are not intended to be limiting or represent an exhaustive enumeration of all aspects of the invention. The scope of the invention is to be determined from the claims appended hereto. Further, it will be apparent to those of skill in the art that numerous changes may be made in such details without departing from the spirit and the principles of the invention.

What is claimed is:

1. A high water oral care composition comprising:
an orally acceptable vehicle;
a thickening system;
one or more abrasives; and
one or more gelling agents,
wherein the one or more gelling agents comprise a salt of a C₁₂-C₃₂ fatty acid; and
wherein the salt is a monovalent salt, a divalent salt, or a combination thereof.

2. (canceled)

3. The oral care composition according to claim 1, wherein the fatty acid is a saturated fatty acid.

4. The oral care composition according to claim 1, wherein the fatty acid is an unsaturated fatty acid.

5. The oral care composition according to claim 4, wherein the unsaturated fatty acid is a C₁₆₋₁₈ fatty acid.

6. The oral care composition according to claim 1, wherein the fatty acid is selected from lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, linoleic acid, arachidonic acid, palmitoleic acid, oleic acid, and a combination of two or more thereof.

7. (canceled)

8. (canceled)

9. The oral care composition according to claim 1, wherein the monovalent salt of a fatty acid is selected from sodium palmitate, sodium stearate, potassium palmitate, potassium stearate, and a combination of two or more thereof.

10. The oral care composition according to claim 1, wherein the one or more gelling agents are present in an amount of from about 0.5 weight % to about 4.5 weight %, based on the total weight of the oral care composition.

11. The oral care composition according to claim 1, wherein the orally acceptable vehicle is selected from glycerin, sorbitol, xylitol, propylene glycol, polyethylene glycol, and combinations of two or more thereof, and wherein the orally acceptable vehicle is present in an amount of from about 50 weight % to about 70 weight %, based on the total weight of the oral care composition.

12. (canceled)

13. The oral care composition according to claim 1, wherein the thickening system comprises colloidal silica, fumed silica, carrageenan, sodium carboxymethylcellulose, or a combination thereof.

14. The oral care composition of claim 13, wherein the thickening system is present in an amount of from about 2 weight % to about 5 weight %, based on the total weight of the oral care composition.

15. The oral care composition according to claim 1, wherein the one or more abrasives comprise calcium salt, silica, or sodium metaphosphate wherein the one or more abrasives are present in an amount of from about 15 weight % to about 25 weight %, based on the total weight of the oral care composition.

16. The oral care composition according to claim 15, wherein the calcium salt is selected from tricalcium phosphate, hydroxyapatite, dicalcium phosphate dihydrate, anhydrous dicalcium phosphate, calcium pyrophosphate, natural calcium carbonate, precipitated calcium carbonate, and mixtures thereof.

17. (canceled)

18. The oral care composition according to claim 1, wherein the oral care composition further comprises from about 0.01 weight % to about 5.0 weight %, based on the total weight of the oral care composition, of a fluoride ion source wherein the fluoride ion source is selected from stannous fluoride, sodium fluoride, potassium fluoride, sodium monofluorophosphate, fluorosilicate salts, such as sodium fluorosilicate and ammonium fluorosilicate, amine fluoride, ammonium fluoride, and combinations thereof.

19. (canceled)

20. (canceled)

21. The oral care composition according to claim 1, wherein the oral care composition is substantially free of fatty alcohols.

22. (canceled)

23. The oral care composition according to claim 1, wherein the oral care composition comprises water in an amount of from about 20 weight % to about 50 weight %, based on the total weight of the oral care composition.

24. The oral care composition according to claim 1, wherein the oral care composition further comprises from about 1% to about 5% by weight, based on the total weight of the oral care composition, of one or more surfactants selected from anionic surfactants; non-ionic surfactants; amphoteric surfactants; and a combination of two or more thereof.

25. (canceled)

26. The oral care composition according to claim 24, wherein the anionic surfactant is selected from sodium lauryl benzene sulfonate, dodecyl sodium sulfoacetate, N-2-ethyl laurate potassium sulfoacetamide), sodium lauryl sarcosinate, sodium lauryl sulfate, sodium ether lauryl sulfate, and combinations thereof.

27. The oral care composition according to claim 26, wherein the amphoteric surfactant comprises a betaine surfactant.

28. The oral care composition according to claim 24, wherein the one or more surfactants is selected from sodium lauryl benzene sulfonate, dodecyl sodium sulfoacetate, N-2-ethyl laurate potassium sulfoacetamide), sodium lauryl sarcosinate, sodium lauryl sulfate, sodium ether lauryl sulfate, cocamidopropyl betaine, and combinations thereof.

29. (canceled)

30. A method for:
cleaning an oral cavity surface;
whitening a tooth;
treating, preventing or ameliorating a symptom associated with gingivitis;
treating, preventing or ameliorating a symptom associated with periodontitis;
freshening breath and/or mitigating oral malodor;
comprising contacting an oral cavity surface of a subject in need thereof, with an oral care composition according to claim 1.
